(19) United States
(12) Patent Application Publication
(10) Pub. No.: US 2021/0386590 A1
(43) Pub. Date: Dec. 16, 2021

(54) DISPOSABLE SINGLE USE EYE SHIELD

(71) Applicant: Christine Josephine SUESS, Cornwall (CA)

(72) Inventor: Christine Josephine SUESS, Cornwall (CA)

(21) Appl. No.: 16/946,246

(22) Filed: Jun. 12, 2020

(51) Int. Cl. A61F 9/04; A61B 3/024

(52) U.S. Cl. CPC A61F 9/04; A61B 3/024

(57) ABSTRACT

Single use, disposable eye shields will eliminate potential transmission of an infectious agent such as COVID to another patient or a technician, nurse or doctor. The product can be made with inexpensive materials readily available—card stock or translucent pliable plastic and elastic thread. An elastic thread can be used to secure the eye shield in place and thereby allow the eye shield to fit all head sizes. All components of the eye shields may be made from biodegradable components. This eye shield does not have any adhesive, so it can be used for the other eye when performing visual field testing for each eye separately. The translucent version has the added benefit of preventing the patient from dark adapting during the visual field test.

### FIELD OF THE INVENTION

The present disclosure relates to an eye shield more specifically, but not by way of limitation, a disposable eye shield and more particularly to a disposable eye shield for use during eye examination procedures.

### BACKGROUND

Perimetry or visual field testing measures all areas of a patient's eyesight, including side, or peripheral, vision. A perimetry test can help find certain patterns of vision loss and determine if certain types of eye disease are present. It is used for early detection of vision changes caused by nerve damage from glaucoma. Regular perimetry tests can be used to see if treatment for glaucoma is preventing further vision loss. During the visual field one eye is tested at a time, requiring the other eye to be shielded in order to accurately perform the test.

There are numerous different types of eye shields available, some are single use and others are designed to be multiple use. Most of the single use shields have adhesive strips to keep them in place, which not only make them expensive, but renders them not suitable for the elderly or patients having fragile or easily irritated skin. The multiple use shields need to be able to be thoroughly cleansed or sterilized after use.

With the advent of COVID-19 and other communicable diseases, the safety of staff charged with cleansing the multiple use shields must now be factored into purchasing decisions.

US20110034849 discloses a disposable self-adhering covering for an eye of a human comprising an opaque, thin, flexible, air permeable fabric layer having an upper surface and a lower surface bonded to top side of a less flexible generally ovoid-shaped generally flat ring defining a central opening. The ring comprising a bottom side comprising an adhesive that allows the covering to be removably attachable to the skin. Alternatively, the invention may comprise a band extending from opposite outer edges of the ring to circle the head to provide additional secure placement of the patch rather than the adhesive.

CN204050006 discloses a single use disposable eye safety shield, comprising a several annular layers linked an elastic webbing. Non-woven fabrics prevents cross infection, and ensures safety using eye equipment and safety of medical personnel and of the patient.

U.S. Pat. No. 4,793,002 discloses adjustable, disposable eye protectors formed from an adhesive-backed ovoid to circular shaped film segment. The conical shaped eye protectors are easy to use for reducing exposure of the eyes to incident light or other potential eye irritants. The segment could be totally opaque, or substantially transparent.

U.S. Pat. No. 10,632,016 discloses a shield for protecting the eye of a patient who is undergoing treatment of a facial area, such as the nose-bridge, forehead, temple, or an area immediately surrounding the eye. The invention relates to a disposable eye patch/shield used during surgical and/or professional facial care procedures. The shell is formed in any suitable manner to the desired shape, preferably a rounded oval, to fit within the eye socket of a patient to be treated but of a sufficient size to always cover the patient's eye and have a curvature that can vary depending upon the desired characteristics and uses for the eye shield.

US20170266053 discloses a disposable eye patch comprising a rim member circumferentially attached to the second layer, wherein the rim member comprises an adhesive for sticking the eye patch to cover the human eye. The adhesive is a safe for contact with the human skin and can be removed and discarded after a single use. The disposable eye patch further comprises an absorbent layer attached to the first layer facing the outer surface of the eye.

EP1886655 discloses a disposable eye patch including at least one sheet member and a light-occluding layer such as a vinyl layer attached to the sheet member. The eye patch may include at least two sheet members, with the light-occluding layer interposed between the sheet members. The eye patch preferably has adhesive on a peripheral portion of the eye patch for attaching the eye patch to a wearer's face.

US20150085257 discloses devices for occluding an eye and, more particularly, to disposable eye patches. The eye patches include a single layer substrate comprising a sheet of material having a first region and a second region. An outer edge extends around the substrate and a slit extends through the substrate so as to form a first flap and a second flap. A first adhesive portion is applied to the substrate near the first region such that the first and second flaps each receive part of the first adhesive portion. A second adhesive portion is applied to the substrate near the second region, and the second adhesive portion is discontinuous with the first adhesive portion. The first and second flaps are configured to be placed into an overlapping arrangement and adhered together by the first adhesive portion to give the eye patch a generally cupped shape. The first and second adhesive portions provide two locations for adhering the eye patch a patient's face.

U.S. Pat. No. 10,517,764 discloses a protective eye cover system comprising a nonporous dome-shaped lens shaped to fit over a human eye cavity and encircle an associated eye. The eye covers each comprise a dome shaped lens attached to a base ring which may be strategically shaped to conform to a typical eye socket comfortably. The lens and base ring may be made of a moldable transparent material and may be thin, non-collapsible, and able to keep its form. Furthermore, it may be desirable for the material to be lightweight, some embodiments may be intended for one-time usage and then disposal while others may be intended to offer multiple usages per lens.

U.S. Pat. No. 9,157,38 discloses eye shields or protectors, and it provides a device which will fit close to the face of the user without exerting any pressure upon, or restricting the movements of the eyeball. The device provides a device which can be adjusted to prevent the admission of light to the eye without utilizing elastics.

U.S. Pat. No. 5,183,059 discloses an eye shield retention system holds a generally ovate eye cover in position across the bony orbital rim of a user's eye. The eye shield comprises a plastic shell adapted to function as an eye shield or as a backup member for an eyelid compress. The retention system has a first strap segment that attaches to the lower end of the eye shield and joins with a second strap segment behind the user's head. The ovate shell is preferably made from a plastic material or a light weight metal such as aluminum. The shell has a slightly convex exterior surface and a slightly concave interior surface and is of substantially uniform thickness providing a space between its interior surface and the eye of the user.

None of the above cited documents, alone or in combination, disclose a disposable eye shield and more particularly a disposable eye shield for use during eye examination procedures. Hence there remains a need for a single patient use version of an eye shield that can be used during visual field testing.

All documents referenced above are incorporated herein in their entirety.

### BRIEF SUMMARY OF THE INVENTION

It is the object of the present invention to provide a disposable single use eye shield.

In accordance with an aspect of the invention, there is provided a single use disposable eye shield, comprising: a shield having an approximately ovate peripheral shape, said shield having inner surface and an outer surface that form a contoured shape, said shield having first and second sides positioned at distal edges of the ovate shape; a first attachment point situated on the first side of said shield; a second attachment point situated on the second side of said shield; and one or more attachment members having first and second ends, said first end of said one or more attachment members attached to said first attachment point and said second end of said one or more attachment members attached to said second attachment point, wherein the one or more attachment members extends from the first attachment point on the first side of said shield around the head of a patient to the second attachment point on the second side of said shield.

In accordance with an embodiment of the invention, there is provided a method of shielding a patient's eye during visual field testing, comprising the steps of: positioning the shield of the single use disposable eye shield over the eye to be shielded; and positioning the one or more attachment members on the patient's head, thereby securing said shield in place over the patient's eye.

The advantages and features of the present invention will become better understood with reference to the following more detailed description and claims taken in conjunction with the accompanying drawings in which like elements are identified with like symbols.

To easily identify the discussion of any particular element or act, the most significant digit or digits in a reference number refer to the figure number in which that element is first introduced.

### BRIEF DESCRIPTION OF THE SEVERAL VIEWS OF THE DRAWINGS

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

FIG. 1 illustrates a perspective view 100 in accordance with one embodiment of the invention.

FIG. 2 illustrates a front view 200 in accordance with one embodiment of the invention.

FIG. 3 illustrates a side view 300 in accordance with one embodiment of the invention.

FIG. 4 illustrates a top view 400 in accordance with one embodiment of the invention.

FIG. 5 illustrates an applied view 500 in accordance with one embodiment of the invention.

### DETAILED DESCRIPTION

Various embodiments of the invention relate to a disposable (single patient use) version of an eye shield that is widely used during visual field testing. Certain embodiments of the invention relate to biodegradable eye shields. Current eye shields that exist are typically reusable and non-biodegradable. Known eye shields are not easy to thoroughly disinfect between patients. Having a disposable version of an eye shield will serve an unmet need for better infection control in medical offices to keep patients and medical staff safe.

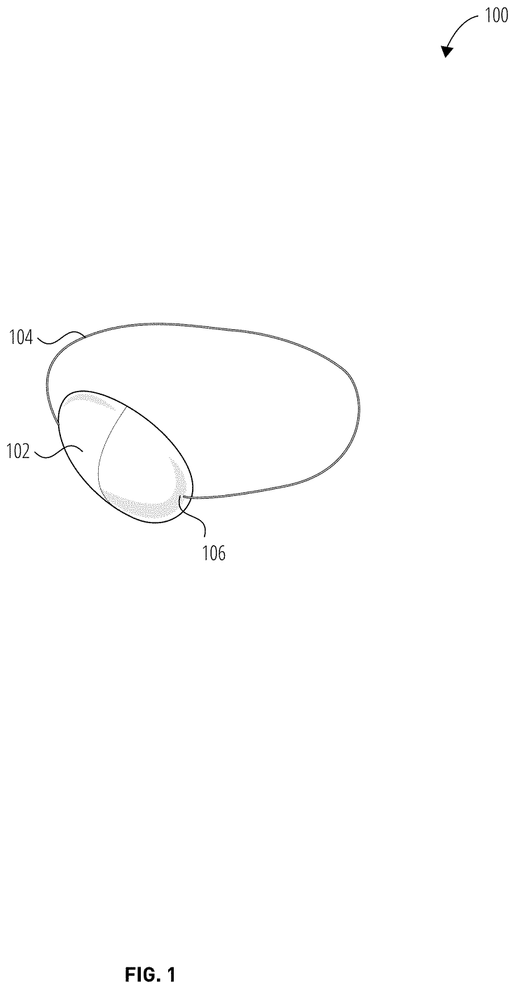

FIG. 1 illustrates a perspective view of an embodiment of the invention. The shield 102 is connected to the attachment member 104 at side of shield with attachment point 106. The embodiment of the invention shown in FIG. 1 is of an essentially ovate shield 102, having a convex front edge. An attachment member 104 is used to attach the disposable eye shield to the patient. The attachment member 104 can be made of either an extensible material such as elastic, spandex or elastane, or of a non-extensible material such as ribbon, thread, twine or the like. When non-extensible materials are used, it is envisioned that the attachment member 104 may be in two separate pieces that can be tied behind the head of the patient.

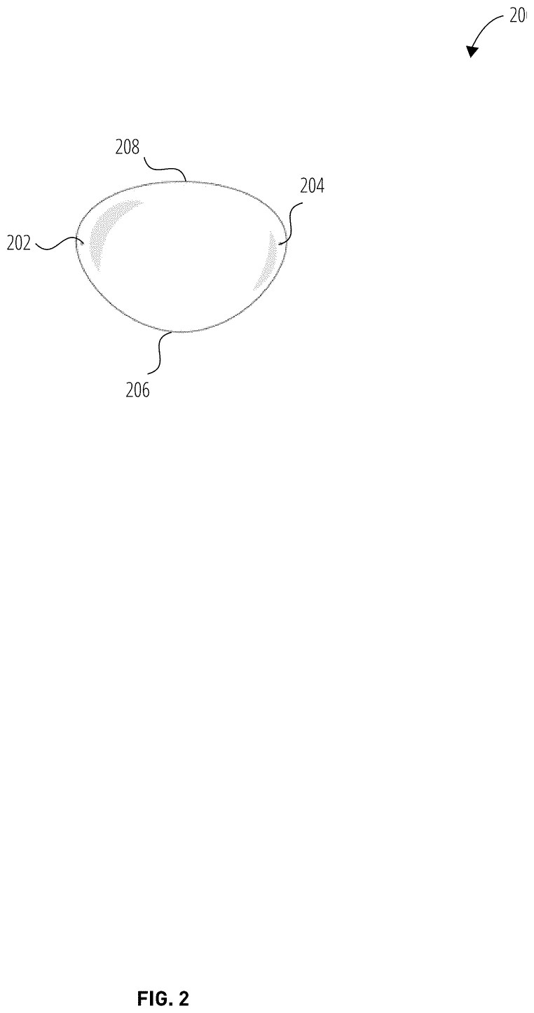

FIG. 2 illustrates a front view of an embodiment of the invention. It can be seen that the shield 102 has two attachment points, the first attachment point 202 on one side of the shield 102 and a second attachment point 204 on the other side. The shield has an upper perimeter of shield 208 that engages with the eyebrow/forehead part of the face, and a lower perimeter of shield 206 which engages with the cheek region of the patient's face.

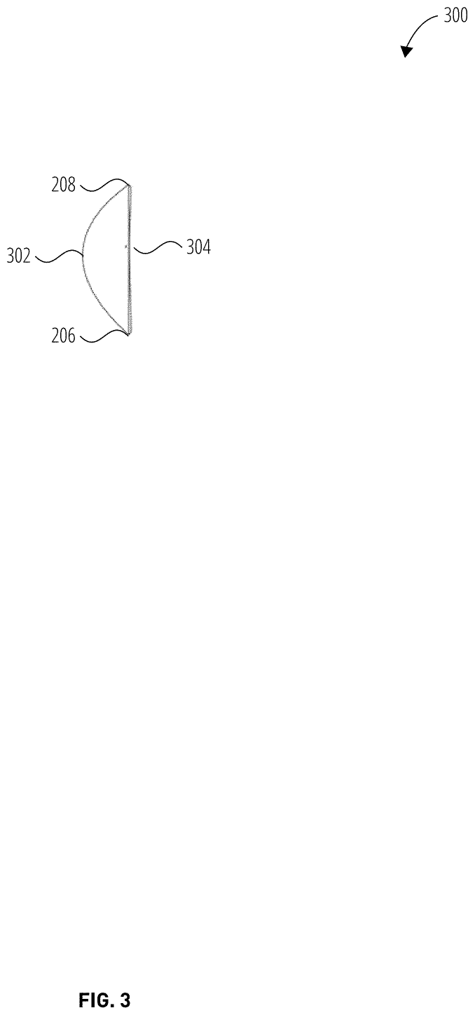

FIG. 3 illustrates a side view of an embodiment of the invention. It can be seen that the shield 102 has a convex outer front edge of shield 302. The curvature renders the shield more comfortable as the inner surface of the shield 102 allows the eye to move freely and the eyelid to open and close unhindered, not touching the eyelashes of the patient.

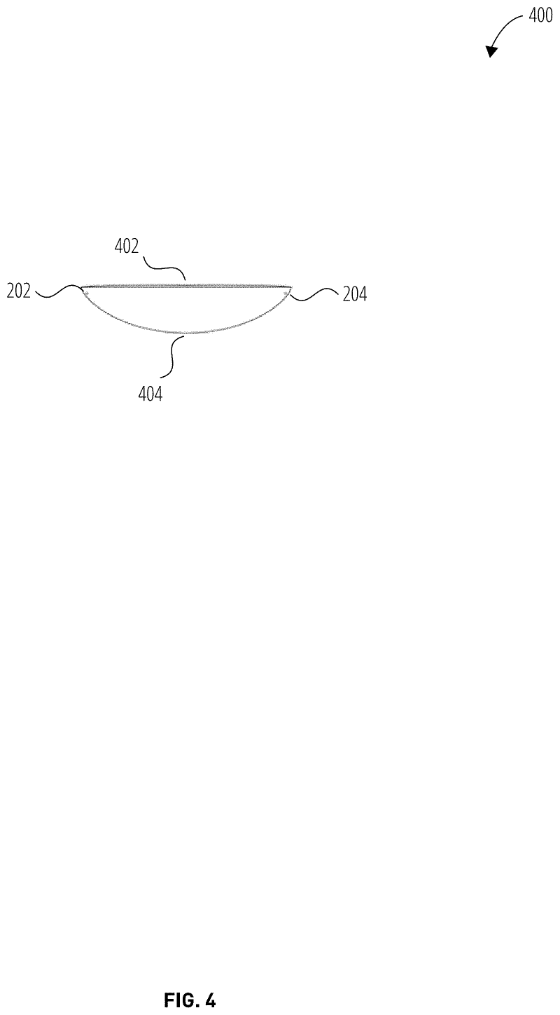

FIG. 4 illustrates a top view of an embodiment of the invention. It can be seen that the shield 102 has a symmetrical shape which enables the shield 102 to be used on either the right or left eye of the patient.

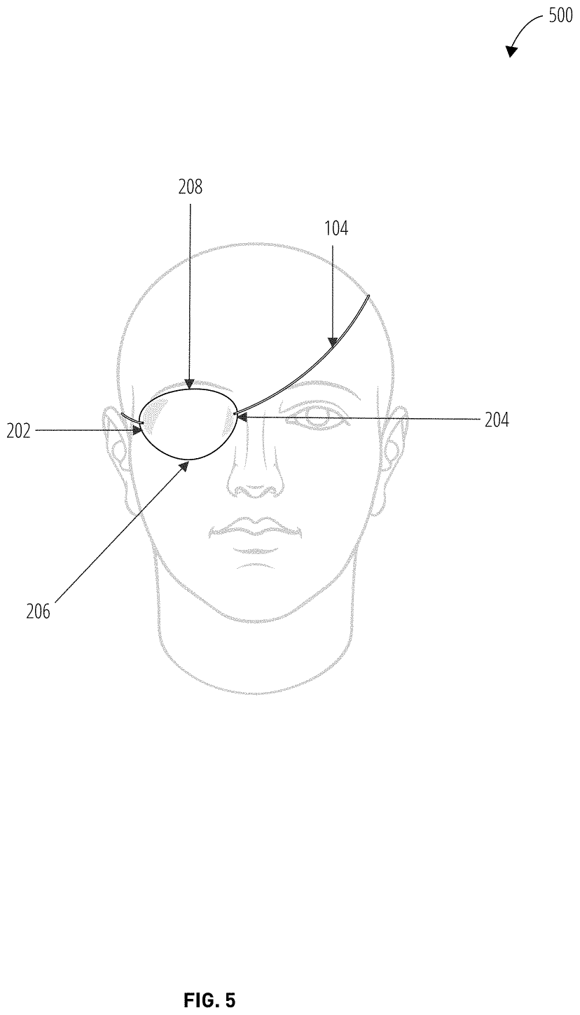

FIG. 5 illustrates an applied view of an embodiment of the invention. It can be seen that the shield 102 can be positioned comfortably on the face of a patient. The attachment member 104 can then be positioned in the most appropriate orientation for the patient.

Embodiments of the eye shields of the current invention are intended as single use, eliminating potential transmission of an infectious agent such as COVID to another patient or even a technician or doctor. The product can be made with inexpensive materials readily available—card stock or translucent pliable plastic and elastic thread, cotton or jute. The thread cotton or jute can be used to secure the eye shield in place and thereby allow the eye shield to fit all head sizes. This eye shield does not have any adhesive, so it can be used for the other eye of the same patient when performing visual field testing for each eye separately. The translucent version has the added benefit of preventing the patient from dark adapting during the visual field test.

The eye patch is very easy to use. They eye shield can be given to the patient to place on their eye themselves. Eliminating the need for the technician or doctor touching the patients face and head in many cases. This allows the technician to spend a shorter period of time in close proximity to the patient and move to a safe social distance quicker.

The eye shield is substantially spherical in plain view but has a concave contour when viewed from the rear. The concave nature of the shield provides room for the subject's eye to move freely, and the eyelid to open and close unhindered when blinking. The shield is, therefore, very comfortable to wear, such that the patient is happy to wear it for extended periods of time, thereby improving the testing procedure.

In one embodiment, the eye shield made from a moldable transparent material such as, but not limited to, polyethylene, polypropylene, polycarbonate, other types of moldable plastics, acrylic materials, and the like. The shield may be opaque and so does not allow any light to permeate there through at any point.

In an alternative embodiment, the eye shield is made from a non-moldable material such as cardstock, cardboard or compressed foam.

In yet an additional embodiment, when using cardstock, cardboard or compressed foam, a generally ovoid to circular shape is selected for the shield. A scission line extending radially inwardly from a point on the edge of the circular shape toward the center of the shape is either folded or cut. A contact adhesive can then be applied essentially along the scission line to produce a conical configuration.

In an alternative embodiment a translucent cover may be employed to render the shield suitable for preventing the patient from dark adapting during the visual field test. The 3D cone shape or relatively circular 2D shape requires no assembly beyond positioning the apparatus to the patient's eye. The components are disposable & one-size fits all.

Another embodiment of the invention, the shield portion is approximately 75 mm oval concave cone shape. It can be white or translucent and made of lightweight cardstock. Attached to thin elastic cord that can secure the patch around the head.

In one alternative embodiment of the invention, the attachment member or members may be tied to the shield through holes present at either side of the shield. The attachment member or members may be tied, for example, with a single, double or slip knot. In a further embodiment of the invention, the attachment member or members may be secured to the shield via a small smooth fastener, grommet or snap.

As it is contemplated that the eye shields of the present invention are single use, various embodiments of the invention may be made from biodegradable components. The attachment members can be made from cotton thread or jute and the shield can be made from paper, card or biodegradable plastic.

Biodegradable plastics decompose faster than the usual plastic as they contain substances that attract microbes to decompose them. Moreover, when they decompose, they do not release harmful gases like carbon dioxide, nitrous oxide and methane into the environment.

Essentially, there are three main types of biodegradable plastics: oxo-biodegradable; hydro-biodegradable and photo-biodegradable plastics. Oxo-biodegradable plastics are currently made from naphtha, which is a by-product of oil refining. The time it takes for oxo-biodegradable products to degrade can be ‘programmed’ at the time of manufacturing process. It takes several months to a year to degrade, but does not release harmful gases such as methane or nitrous oxide. Hydro-biodegradable plastic is made from plant sources such as starch and the degradation is initiated by hydrolysis. These types of plastic degrade faster than oxo-biodegradable plastic and can be industrially composted. They are temperature resistant, and the only bioplastic that will decompose in both soil and water. Polylactic acid or PLA is the most commonly used bio-degradable plastic and it also does not emit potentially harmful methane gas at any stage. Photo-degradable plastic reacts to ultra-violet light, however it does require initial oxo-degradation prior to successful degradation in the environment.

All eye shields are to be made in a clean environment. Some indications and usages may require that the shields are individually packaged. Furthermore, some usages may require that the eye shields are sterile. Numerous components, including but not limited to biodegradable plastics, can be heat sterilized and other materials known to those in the filed can be sterilized by gamma irradiation.

The foregoing descriptions of specific embodiments of the present invention have been presented for purposes of illustration and description. They are not intended to be exhaustive or to limit the invention and method of use to the precise forms disclosed. Obviously, many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient but is intended to cover the application or implementation without departing from the spirit or scope of the claims of the present invention.

What is claimed is:

1. A single use disposable eye shield, comprising:
a shield having an approximately ovate peripheral shape, said shield having inner surface and an outer surface that form a contoured shape, said shield having first and second sides positioned at distal edges of the ovate shape;
a first attachment point situated on the first side of said shield;
a second attachment point situated on the second side of said shield; and
one or more attachment members having first and second ends, said first end of said one or more attachment members attached to said first attachment point and said second end of said one or more attachment members attached to said second attachment point,
wherein the one or more attachment members extends from the first attachment point on the first side of said shield around the head of a patient to the second attachment point on the second side of said shield.

2. The disposable eye shield of claim 1, wherein said contoured shape is outwardly convex.

3. The disposable eye shield of claim 1, wherein the shield is composed of a moldable transparent material selected from polyethylene, polypropylene, polycarbonate, moldable plastics or acrylic materials.

4. The disposable eye shield of claim 1, wherein the shield is composed biodegradable plastic selected from the group comprising oxo-biodegradable; hydro-biodegradable and photo-biodegradable plastic.

5. The disposable eye shield of claim 1, wherein the shield is composed of cardstock, cardboard or compressed foam.

6. The disposable eye shield of claim 1, wherein said first and second attachment points are first and second holes punched through the first and second sides of said shield.

7. The disposable eye shield of claim 1, comprising one attachment member.

8. The disposable eye shield of claim 7, wherein said first end of said attachment member is tied to the first hole and said second end of said attachment member is tied to the second hole.

9. The disposable eye shield of claim 1, comprising two attachment members.

10. The disposable eye shield of claim 9, wherein said first end of said first attachment member is tied to the first hole and said second end of said second attachment member is tied to the second hole.

11. The disposable eye shield of claim 10, wherein said first and second attachment members are tied together at a length to fit the patient's head.

12. The disposable eye shield of claim 1, wherein said attachment member is comprised of an extensible material.

13. The disposable eye shield of claim 1, wherein the extensible material is selected from elastic, spandex or elastane.

14. The disposable eye shield of claim 1, wherein said attachment member is comprised of a non-extensible material.

15. The disposable eye shield of claim 1, wherein said non-extensible material is selected from ribbon, thread, cotton, jute or twine.

16. The disposable eye shield of claim 1, wherein said shield is opaque or translucent.

17. The disposable eye shield of claim 1, wherein the single use disposable shield is manufactured in a clean facility.

18. The disposable eye shield of claim 1, wherein the single use disposable shield is sterile.

19. A method of shielding a patient's eye during visual field testing, comprising the steps of:
positioning the shield of the single use disposable eye shield of claim 1 over the eye to be shielded; and
positioning the one or more attachment members on the patient's head,
thereby securing said shield in place over the patient's eye.

20. The method of claim 19, wherein the single use disposable eye shield comprises two attachment members, and the method additionally comprises the step of tying the two attachment members together at a length to fit the patient's head
